(19) United States
(12) Patent Application Publication
(10) Pub. No.: US 2016/0251279 A1
(43) Pub. Date: Sep. 1, 2016

(54) A PROCESS FOR THE AROMATIZATION OF ALKANES IN AN ALKANE-CONTAINING GAS STREAM

(71) Applicant: SHELL OIL COMPANY, Houston, TX (US)

(72) Inventors: Peter Tanev TANEV, Katy, TX (US); Larry Lanier MARSHALL, Houston, TX (US); Anand NILEKAR, Houston, TX (US); Daniel Edward GERWIEN, Sugar Land, TX (US); Waleed Yousef MUSALLAM, Houston, TX (US); David Morris HAMILTON, JR., Sugar Land, TX (US); Lizbeth Olivia CISNEROS TREVINO, Katy, TX (US); Ann Marie LAURITZEN, JR., Houston, TX (US); Juan Mirabel GARZA, Richmond, TX (US)

(73) Assignee: Shell Oil Company, Houston, TX (US)

(21) Appl. No.: 15/028,832

(22) PCT Filed: Oct. 9, 2014
(86) PCT No.: PCT/US2014/059869

§ 371 Date: Apr. 12, 2016

Related U.S. Application Data
(60) Provisional application No. 61/890,595, filed on Oct. 14, 2013.

(51) Int. Cl. C07C 2/76

(52) U.S. Cl. CPC C07C 2/76; C07C 2529/40; C07C 2529/65; C07C 2529/70

(57) ABSTRACT

A process for the aromatization of alkanes in an alkane-containing gas stream comprising: contacting the alkane-containing gas stream in a reactor with a fluidized bed comprising an aromatization catalyst and a hydrogen acceptor under alkane aromatization conditions to produce a product stream comprising aromatics and hydrogen wherein the hydrogen is, at least in part, bound by the hydrogen acceptor in the reaction zone and removed from the product and the reaction zone.

### FIELD OF THE INVENTION

This invention relates to a process for the aromatization of alkanes in a C₂-C₄ alkane-containing gas stream to form aromatics and hydrogen in a reactor containing both catalyst and hydrogen acceptor particles in a fluidized bed state wherein the removal of hydrogen from the reaction zone is accomplished in-situ by the hydrogen acceptor.

### BACKGROUND OF THE INVENTION

The aromatic hydrocarbons (specifically benzene, toluene and xylenes) are the main high-octane bearing components of the gasoline pool and important petrochemical building blocks used to produce high value chemicals and a variety of consumer products, such as styrene, phenol, polymers, plastics, medicines, and others. Since the late 1930's, aromatics are primarily produced by upgrading of oil-derived feedstocks via catalytic reforming or cracking of heavy naphthas. However, occasional severe oil shortages and price spikes result in severe aromatics shortages and price spikes. Therefore, there is a need to develop new and independent from oil, commercial routes to produce high value aromatics from highly abundant and inexpensive hydrocarbon feedstocks such as natural gas liquids, LPG or associated gas, but also refinery or petrochemical streams including waste streams.

To meet this projected supply shortage, numerous catalysts and processes for on-purpose production of aromatics (including benzene) from alkanes containing six or less carbon atoms per molecule have been investigated. These catalysts are usually bifunctional, containing a zeolite or molecular sieve material to provide acidity and one or more metals such as Pt, Ga, Zn, Mo, etc. to provide dehydrogenation activity. For example, U.S. Pat. No. 4,350,835 describes a process for converting ethane-containing gaseous feeds to aromatics using a crystalline zeolite catalyst of the ZSM-5-type family containing a minor amount of Ga. As another example, U.S. Pat. No. 7,186,871 describes aromatization of C₁-C₄ alkanes using a catalyst containing Pt and ZSM-5.

Despite these efforts, a direct, non-oxidative alkane aromatization catalyst and process cannot yet be commercialized. Some important challenges that need to be overcome to commercialize this process include: (i) the low, as dictated by thermodynamic equilibrium, per pass conversion and benzene yield (for example, ca. 48% wt and 42% wt for ethane dehydroaromatization to benzene at atmospheric pressure, 575° C.); (ii) the fact that the reaction is favored by high temperature and low pressure; (iii) the need to separate the produced aromatics and hydrogen from unreacted hydrocarbon off gas and (iv) the rapid coke formation and deposition on the catalyst surface and corresponding relatively fast catalyst deactivation. Among these challenges, overcoming the thermodynamic equilibrium limitations and significantly improving the conversion and benzene yield per pass has the potential to enable the commercialization of an efficient, direct, non-oxidative alkane aromatization process.

Examples of C₂-C₄ alkane aromatization reactions include the following:

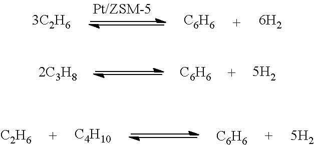

According to the reactions, 2 to 3 alkane molecules are required to generate a molecule of benzene. It is also apparent that, the generation of a molecule of benzene is accompanied by the generation of 5 to 6 molecules of hydrogen. Simple thermodynamic calculations reveal that, for example, ethane dehydroaromatization at atmospheric pressure is equilibrium limited to about 48% ethane conversion at reaction temperature of 575° C. In addition, the equilibrium benzene yield at these conditions is limited to about 42% wt if no side reactions take place. The generation of 6 molecules of hydrogen per molecule of benzene during the ethane dehydroaromatization reaction leads to significant volume expansion that suppresses the reaction to proceed to the right, i.e. it suppresses alkane conversion and formation of reaction products, i.e. benzene yield. The aforementioned low per pass conversions and benzene yields and/or high temperature requirement are not very attractive to provide an economic justification for scale-up and commercialization of alkane dehydroaromatization processes.

Therefore, there is a need to develop an improved direct, non-oxidative lower alkane containing gas stream aromatization process that provides for significantly higher (than allowed by the thermodynamic equilibrium) conversion and benzene yields per pass by implementing an in-situ hydrogen removal from the reaction zone.

### SUMMARY OF THE INVENTION

The invention provides a process for the aromatization of alkanes in an alkane-containing gas stream, which alkane-containing gas stream contains at least one alkane selected from the group consisting of ethane, propane or butane and contains essentially no methane, comprising: contacting the alkane-containing gas stream in a reactor with a fluidized bed comprising an aromatization catalyst and a hydrogen acceptor under alkane-containing gas aromatization conditions to produce a product stream comprising aromatics and hydrogen wherein the hydrogen is, at least in part, bound by the hydrogen acceptor in the reaction zone and removed from the product and reaction zone.

The invention further provides unique process schemes for recycling and regenerating the catalyst and hydrogen acceptor particles wherein both catalyst and hydrogen acceptor particles are continuously withdrawn from the reaction zone of the reactor, fed to a regeneration vessel or vessels to be regenerated and returned back into the reactor for continuous (uninterrupted) production of aromatics and hydrogen. The aforementioned in-situ hydrogen removal in a fluidized bed state allows for overcoming the thermodynamic equilibrium limitations and for shifting the reaction equilibrium to the right. This results in significantly higher and economically more attractive alkane conversion and benzene yields per pass relative to the case without hydrogen removal in the reaction zone.

### BRIEF DESCRIPTION OF THE DRAWINGS

FIG. 1 shows a schematic diagram of an embodiment of the invention: aromatization reactor with catalyst and hydrogen acceptor particles intermixed in a fluidized bed.

FIG. 2 shows a schematic diagram of an embodiment of the invention: co-regeneration of both hydrogen acceptor and catalyst particles in a single vessel.

FIG. 3 shows a schematic diagram of another embodiment of the invention: regeneration of hydrogen acceptor and catalyst particles in separate vessels.

### DETAILED DESCRIPTION

The present application relates to the conversion of alkanes in an alkane-containing gas stream that contains at least one alkane selected from the group consisting of ethane, propane or butane and contains essentially no methane. This gas stream is used as a feed stream to an alkane dehydroaromatization process according to the present invention, wherein at least part of the alkanes are converted to aromatic products, such as benzene toluene and xylenes. Herein below this gas stream is also referred to as the alkane-containing gas stream or the C₂-C₄ alkane-containing gas stream. The conversion of alkenes in the alkane-containing gas stream to aromatics is typically carried out in a reactor comprising a catalyst, which is active in the conversion of the alkanes to aromatics. The alkane-containing gas stream that is fed to the reactor comprises in the range of from 50 to 100% vol. C₂-C₄ alkane, preferably in the range of from 70 to 100% vol. C₂-C₄ alkane and more preferably in the range of from 75% vol. to 100% vol. C₂-C₄ alkane, based on the alkane-containing gas stream. Preferably, the balance of the alkane-containing gas may be other C2+ alkanes, C₂-C₄ olefins, nitrogen carbon dioxide and other non-hydrocarbon gases. The feed may contain small amounts of C₂-C₄ olefins, preferably no more than 5 to 10 weight percent. Too much olefin may cause an unacceptable amount of coking and deactivation of the catalyst. The alkane-containing gas stream may be or be derived from for instance natural gas liquids, LPG or associated gas, but also from refinery or petrochemical streams including waste streams.

Natural gas liquids are produced as part of natural gas and typically consist of ethane and propane. The natural gas liquids may make-up to 30% vol. of a typical natural gas source.

The alkane-containing gas contains essentially no methane, preferably contains no methane. Methane is difficult to convert under C₂-C₄ alkane aromatization conditions and therefore ends up in the product stream. Separation of methane from the other compounds in the product stream is energy consuming process, typically requiring a cold-box type separation. Moreover, as the methane is essentially inert it further undesirably increases the volume of the stream flowing through the reactor.

The alkane-containing gas is preferably comprised of at least 40% vol. of ethane and/or propane and, optionally, at least 10 to 20% vol. of butane, pentane, etc. Preferably, in the range for from 50 to 100% vol. of the alkane in the alkane-containing gas is ethane, more preferably in the range of from 70 to 100% vol. of the alkane in the alkane-containing gas is ethane and more preferably in the range of from 75% vol. to 100% vol. of the alkane in the alkane-containing gas is ethane. As the conversion of ethane to aromatics produces the largest number of moles hydrogen per mole of e.g. benzene, the advantages of the process according to the invention are most prominent when an alkane-containing gas is used that contains a majority of ethane.

An ethane, propane, butane, mixed propane/ethane or mixed C₂-C₄ lower alkane feed stream may be derived from, for example, an ethane/propane-rich stream derived from natural gas, refinery or petrochemical streams including waste streams. Examples of potentially suitable feed streams include (but are not limited to) residual ethane and propane and butane from natural gas (methane) purification, pure ethane and propane and butane streams (also known as Natural Gas Liquids) co-produced at a liquefied natural gas (LNG) site, C₂-C₄ streams from associated gases co-produced with crude oil production (which are usually too small to justify building a LNG plant but may be sufficient for a chemical plant), unreacted “waste” streams from steam crackers, and the C₂-C₄ byproduct stream from naphtha reformers (the latter two are of low value in some markets such as the Middle East).

Usually natural gas, comprising predominantly methane, enters an LNG plant at elevated pressures and is pre-treated to produce a purified feed stock suitable for liquefaction at cryogenic temperatures. Ethane, propane, butane and other gases are separated from the methane. The purified gas (methane) is processed through a plurality of cooling stages using heat exchangers to progressively reduce its temperature until liquefaction is achieved. The separated gases may be used as the feed stream of the present invention. The byproduct streams produced by the process of the present invention may have to be cooled for storage or recycle and the cooling may be carried out using the heat exchangers used for the cooling of the purified methane gas.

The conversion of alkanes in the alkane-containing gas stream is preferably carried out at a gas hourly space velocity in the range of from 25 to 10000 h⁻¹, more preferably of from 40 to 8000 h⁻¹, even more preferably of from 70 to 6000 h⁻1. The conversion of alkanes in the alkane-containing gas stream is preferably carried out at a pressure in the range of from 0.1 to 10 bara, more preferably of from 0.5 to 5 bara, even more preferably of from 0.5 to 4 bara. The conversion of alkanes in the alkane-containing gas stream is preferably carried out at a temperature in the range of from 400 to 750° C., more preferably of from 450 to 720° C., even more preferably 480 to 700° C. Various co-feeds or additives such as CO, CO₂, hydrogen, H₂S, H2O or mixtures thereof could be added at levels of <10% vol. to the alkane-containing feed to improve the stability, performance or regenerability of the catalyst. The C₂-C₄ alkane aromatization is then carried out until conversion falls to values that are lower than those that are economically acceptable. At this point, the aromatization catalyst has to be regenerated to restore its aromatization activity to a level similar to its original activity. Following the regeneration, the catalyst is again contacted with an alkane-containing gas stream in the reaction zone of the aromatization reactor under alkane aromatization conditions for continuous production of aromatics.

Any catalyst suitable for C₂-C₄ alkane-containing gas stream aromatization may be used in the process of this invention. The catalyst typically comprises one or more active metals deposited on an inorganic oxide support and optionally comprises promoters or other beneficial compounds. The active metal or metals, promoters, compounds as well as the inorganic support all contribute to the overall aromatization activity, mechanical strength and performance of the aromatization catalyst.

Suitable catalyst are for instance described in U.S. Pat. No. 4,899,006, U.S. Pat. No. 5,227,557, EP0244162, U.S. Pat. No. 7,186,871, U.S. Pat. No. 7,186,872, US20090209795, US2009020995, US20110021853, US20090209794 and U.S. Provisional Patent Application No. 61/739,089 all of which are hereby incorporated by reference.

Any one of a variety of catalysts may be used to promote the reaction of ethane and propane and possibly other alkanes to aromatic hydrocarbons. One such catalyst is described in U.S. Pat. No. 4,899,006 which is herein incorporated by reference in its entirety. The catalyst composition described therein comprises an aluminosilicate having gallium deposited thereon and/or an aluminosilicate in which cations have been exchanged with gallium ions. The molar ratio of silica to alumina is at least 5:1.

Another catalyst which may be used in the process of the present invention is described in EP0244162. This catalyst comprises the catalyst described in the preceding paragraph and a Group VIII metal selected from rhodium and platinum. The aluminosilicates are said to preferably be MFI or MEL type structures, but may be selected from ZSM-5 (MFI), ZSM-8 MFI/MEL), ZSM-11 (MEL), ZSM-12 (MTW) or ZSM-35 (FER).

Other catalysts which may be used in the process of the present invention are described in U.S. Pat. No. 7,186,871 and U.S. Pat. No. 7,186,872, both of which are herein incorporated by reference in their entirety. The first of these patents describes a platinum containing ZSM-5 crystalline zeolite synthesized by preparing the zeolite containing the aluminum and silicon in the framework, depositing platinum on the zeolite and calcining the zeolite. The second patent describes such a catalyst which contains gallium in the framework and is essentially aluminum-free.

It is preferred that the catalyst be comprised of a zeolite, a noble metal of the platinum family to promote the dehydrogenation reaction, and a second inert or less active metal which will attenuate the tendency of the noble metal to catalyze hydrogenolysis of the C₂ and higher hydrocarbons in the feed to methane and/or ethane. Attenuating metals which can be used include those described below.

Additional catalysts which may be used in the process of the present invention include those described in U.S. Pat. No. 5,227,557, hereby incorporated by reference in its entirety. These catalysts contain an MFI zeolite plus at least one noble metal from the platinum family and at least one additional metal chosen from the group consisting of tin, germanium, lead, and indium.

One preferred catalyst for use in this invention is described in US20090209795. This publication is hereby incorporated by reference in its entirety. The publication describes a catalyst comprising: (1) 0.005 to 0.1 wt % (% by weight) platinum, based on the metal, preferably 0.01 to 0.05 wt %, (2) an amount of an attenuating metal selected from the group consisting of tin, lead, and germanium which is preferably not more than 0.2 wt % of the catalyst, based on the metal and wherein the amount of platinum may be no more than 0.02 wt % more than the amount of the attenuating metal; (3) 10 to 99.9 wt % of an aluminosilicate, preferably a zeolite, based on the aluminosilicate, preferably 30 to 99.9 wt %, preferably selected from the group consisting of ZSM-5, ZSM-11, ZSM-12, ZSM-23, or ZSM-35, preferably converted to the H+ form, preferably having a SiO₂/Al₂O₃ molar ratio of from 20:1 to 80:1, and (4) a binder, preferably selected from silica, alumina and mixtures thereof.

Another preferred catalyst for use in this invention is described in US20110021853. This application is hereby incorporated by reference in its entirety. The application describes a catalyst comprising: (1) 0.005 to 0.1 wt % (% by weight) platinum, based on the metal, preferably 0.01 to 0.06 wt %, most preferably 0.01 to 0.05 wt %, (2) an amount of iron which is equal to or greater than the amount of the platinum but not more than 0.50 wt % of the catalyst, preferably not more than 0.20 wt % of the catalyst, most preferably not more than 0.10 wt % of the catalyst, based on the metal; (3) 10 to 99.9 wt % of an aluminosilicate, preferably a zeolite, based on the aluminosilicate, preferably 30 to 99.9 wt %, preferably selected from the group consisting of ZSM-5, ZSM-11, ZSM-12, ZSM-23, or ZSM-35, preferably converted to the H+ form, preferably having a SiO₂/Al₂O₃ molar ratio of from 20:1 to 80:1, and (4) a binder, preferably selected from silica, alumina and mixtures thereof.

Another preferred catalyst for use in this invention is described in US20090209794. This publication is hereby incorporated by reference in its entirety. The publication describes a catalyst comprising: (1) 0.005 to 0.1 wt % (% by weight) platinum, based on the metal, preferably 0.01 to 0.05% wt, most preferably 0.02 to 0.05% wt, (2) an amount of gallium which is equal to or greater than the amount of the platinum, preferably no more than 1 wt %, most preferably no more than 0.5 wt %, based on the metal; (3) 10 to 99.9 wt % of an aluminosilicate, preferably a zeolite, based on the aluminosilicate, preferably 30 to 99.9 wt %, preferably selected from the group consisting of ZSM-5, ZSM-11, ZSM-12, ZSM-23, or ZSM-35, preferably converted to the H+ form, preferably having a SiO₂/Al₂O₃ molar ratio of from 20:1 to 80:1, and (4) a binder, preferably selected from silica, alumina and mixtures thereof.

Still another preferred catalyst comprises is disclosed in U.S. Provisional Patent Application No. 61/739,089, which catalyst comprises from about 0.005 to about 0.09% wt platinum, basis the metal. The platinum is highly active in terms of catalyzing not only the desired dehydroaromatization reaction but also an undesired hydrogenolysis reaction leading to lower-value byproduct methane, so it is best if its concentration in the catalyst not be more than 0.1% wt because otherwise too much methane will be produced. In one embodiment from about 0.005 to about 0.05% wt of platinum is used.

An attenuating metal or metals may also be added to the catalyst of the present invention. While the attenuating metal may have catalytic activity in its own right, its main function is to moderate the catalytic activity of platinum so as to reduce the production of less-valuable methane byproduct. Examples of suitable attenuating metals include but are not limited to tin, lead, germanium, and gallium. The attenuating metal comprises not more than about 0.5% wt of the catalyst, basis the metal, more preferably not more than about 0.2% wt and most preferably not more than about 0.1% wt of the attenuating metal is utilized because more than that can cause the overall conversion to aromatics to become too low for commercial use.

The catalyst may comprise boron in an amount of less than 1 wt %. Preferably, if present the amount of boron may be in the range of from 0.005 to 1 wt %, preferably of from 0.01 to 0.6 wt %, more preferably of from 0.02 to 0.4 wt %, based on the weight of the catalyst.

The catalyst also comprises from about 10 to about 99.9% wt of one or more aluminosilicate materials, preferably from about 30 to about 99.9% wt, basis the aluminosilicate(s). The aluminosilicates preferably have a silicon dioxide:aluminum trioxide (SiO₂:Al₂O₃) molar ratio of from about 20 to about 80. The aluminosilicates may preferably be zeolites having the MFI or MEL type structure and may be ZSM-5, ZSM-8, ZSM-11, ZSM-12 or ZSM-35. The zeolite or zeolite mixture is preferably converted to H+ form to provide sufficient acidity to help catalyze the dehydroaromatization reaction. This can be accomplished by calcining the ammonium form of the zeolite in air at a temperature of at least about 400° C.

The final shaped catalyst could be in the form of cylindrical pellets, rings or spheres. The preferred catalyst shape of this invention (for fluidized bed reactor operation) is spherical. The spherical catalyst of this invention could be prepared by any method known to those skilled in the art. Preferably, the spherical catalyst of this invention is prepared via spray drying of zeolite containing sols of appropriate concentration and composition. The zeolite containing sol may optionally contain binder. The spherical catalyst has predominant particle size or diameter that makes it suitable for fluidization. The spherical particle diameter of the catalyst of this invention is preferably selected to be in the range of 20-500 microns. More preferably, the spherical catalyst of this invention has particle diameter in the range of 50-200 microns.

The hydrogen acceptor used in this reaction can be any metal-containing alloy or a compound that has the ability, when subjected to alkane aromatization operating conditions, to selectively accept or react with hydrogen to form a sufficiently strong hydrogen-acceptor bond. The hydrogen acceptor preferably reversibly binds the hydrogen in such a way that during operation in the fluidized bed reactor the hydrogen is strongly bound to the acceptor under the alkane aromatization conditions. In addition, the hydrogen acceptor is preferably able to release the hydrogen when transported to the regeneration section where it is subjected to a different set of (regeneration) conditions that favor release of the previously bound hydrogen and regeneration of the hydrogen acceptor.

Suitable hydrogen acceptors include: Ti, Zr, V, Nb, Hf, Co, Mg, La, Pd, Ni, Fe, Cu, Ag, Cr, Th as well as other transition metals, elements or compounds or mixtures thereof. The hydrogen acceptor may comprise metals that exhibit magnetic properties, such as for example Fe, Co or Ni or various ferro-, para- or diamagnetic alloys of these metals. One or more hydrogen acceptors that exhibit appropriate particle sizes and mass for fluidized bed aromatization operation may be used in the reaction zone to achieve the desired degree of hydrogen separation and removal.

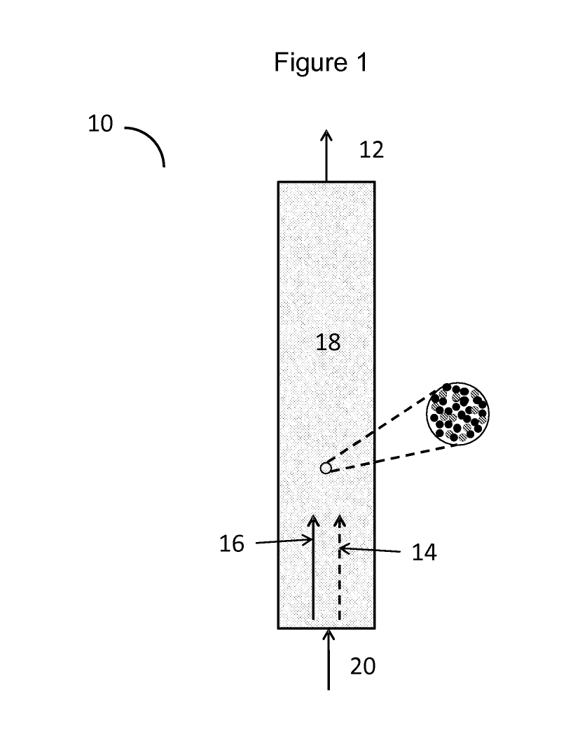

The aromatization reaction of this invention is carried out in a fluidized bed reactor. To enable this, suitably shaped and sufficiently robust catalyst and hydrogen acceptor particles that are able to sustain the rigors of high severity fluidized-bed operation under alkane aromatization reaction conditions are prepared and used for the reaction. According to the present invention, the use of the catalyst and hydrogen acceptor in a fluidized bed reactor and configuration provides several important advantages over the prior art. The most significant advantage of the process of this invention is that it provides for in-situ removal of hydrogen from the reaction zone and as a consequence, significant increase of both alkane conversion and benzene yield per pass to values that are significantly higher relative to these dictated by the alkane aromatization reaction equilibrium. This is enabled by mixing and placing the catalyst and hydrogen acceptor particles in a fluidized-bed state in the reaction zone or the aromatization reactor (see FIG. 1). In FIG. 1, a fluidized bed reactor 10 comprises a mixture of catalyst and hydrogen acceptor particles in the fluidized bed 18. The alkane-containing gas stream, the catalyst and hydrogen acceptors are introduced via one or more inlets (20) and the products, unreacted gases, catalyst and hydrogen acceptor are removed from the bed via one or more outlets 12. The feed and products flow upward in the direction of arrow 16. The catalyst and hydrogen acceptor are introduced upwardly in the direction of arrow 14 (although the catalyst and hydrogen acceptor then form a fluidized bed)

The mixing of both types of particles in a fluidized bed state provides for the quick removal of the produced hydrogen from the reaction zone and for shifting the aromatization reaction equilibrium toward greater alkane-containing gas conversion and benzene yields per pass. Another advantage of the present invention is that it allows, under fluidized bed operating conditions, for volume expansion of the hydrogen acceptor particles during the process of binding of hydrogen to take place. Hydrogen acceptors undergo significant volume expansion in the process of binding hydrogen and at some point in the process the hydrogen acceptor will bind so much hydrogen that it reaches its maximum hydrogen binding capacity. If the acceptor were used in a fixed bed reactor configuration it would expand and agglomerate in the confined bed volume. This would cause agglomeration of the hydrogen acceptor particles, plugging and significant reactor pressure drop, and suppression of the aromatization reaction.

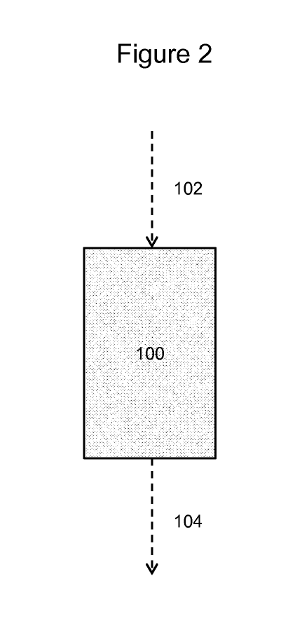

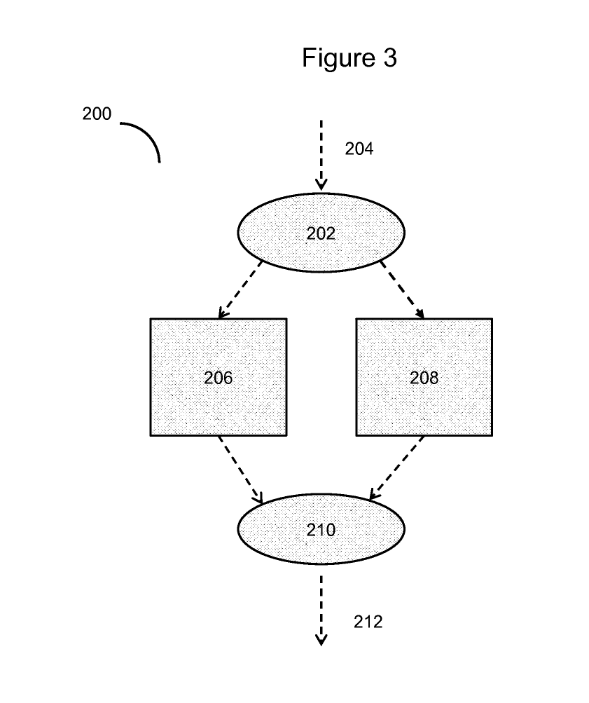

Yet another advantage of the present invention is that, the particle shapes, sizes and mass of both hydrogen acceptor and catalyst particles are designed and selected in such a way so that they could be co-fluidized in the reactor to form the desired fluidized bed. Also, the invention provides for two or more different by chemical formula and/or physical properties hydrogen acceptors to be simultaneously used with the catalyst in the fluidized bed reactor to achieve the desired degree of hydrogen separation from the aromatization reaction zone. Another important advantage of the process of this invention is that it provides for the catalyst and the hydrogen acceptor particles to be simultaneously and continuously withdrawn from the reaction zone or the reactor, regenerated in separate vessel or vessels according to one of the schemes illustrated in FIGS. 2 and 3 and then continuously returned back to the reactor for continuous aromatics and hydrogen production. The hydrogen acceptor and catalyst regeneration could be accomplished either simultaneously or stepwise in the same vessel as illustrated in FIG. 2 or separately in separate vessels as illustrated in FIG. 3. This later operation scheme provides for maximum flexibility to accomplish the hydrogen release or regeneration of the acceptor and catalyst under different and suitable for the purpose set of operating conditions. The regeneration of catalyst and hydrogen acceptor could be accomplished in fixed, moving or fluidized bed reactor vessels schematically shown in FIGS. 2 and 3.

In FIG. 2, regenerator vessel 100 is used to regenerate the catalyst and regenerate the hydrogen acceptor. The catalyst and hydrogen acceptor particles are introduced via inlet 102 and are then removed via outlet 104. Hydrogen removed from the hydrogen acceptor and gases produced by catalyst regeneration are removed from the regenerator via one or more outlets (not shown).

In FIG. 3, regenerator comprises a separation step 202 to separate the catalyst from the hydrogen acceptor that is fed from the reactor via line 204. The catalyst is fed to catalyst regeneration vessel 206, and the hydrogen acceptor is fed to hydrogen acceptor regeneration vessel 208. The catalyst and hydrogen acceptor are then mixed back together in mixing step 210 and then fed back to the reactor via line 212.

In the case of separate regeneration (see FIG. 3), the hydrogen acceptor particles could be separated from the catalyst on the basis of (but not limited to) differences in mass, particle size, density or on the basis of difference in magnetic properties between the acceptor and the catalyst particles. In the latter case, the hydrogen acceptor of this invention could be selected from the group of materials exhibiting fero-, para-or diamagnetic properties and comprising Fe, Co or Ni. It is well known that, the alkane-containing gas aromatization catalysts form coke during the reaction. Accumulation of coke on the surface of the catalyst gradually covers the active aromatization sites of the catalyst resulting in gradual reduction of its activity.

Therefore, the coked catalyst has to be removed at certain carefully chosen frequencies from the reaction zone of the aromatization reactor and regenerated in one of the regeneration vessels depicted in FIGS. 2 and 3. The regeneration of the catalyst can be carried out by any method known to those skilled in the art. For example, two possible regeneration methods are hot hydrogen stripping and oxidative burning at temperatures sufficient to remove the coke from the surface of the catalyst. If hot hydrogen stripping is used to regenerate the catalyst, then at least a portion of the hydrogen used for the catalyst regeneration may come from the hydrogen released from the hydrogen acceptor. Additionally, fresh hydrogen may be fed to the catalyst regeneration vessel as needed to properly supplement the hydrogen released from the hydrogen acceptor and to complete the catalyst regeneration. If the regeneration is carried out in the same vessel (see FIG. 2), then the hydrogen removed from the hydrogen acceptor in-situ could at least partially hydrogen strip and regenerate the catalyst. If the regeneration is carried out in different vessels (see FIG. 3) the operating conditions of each vessel could be selected and maintained to favor the regeneration of the catalyst or the hydrogen acceptor. Hydrogen removed from the hydrogen acceptor could then again be used to at least partially hydrogen strip and regenerate the catalyst.

Yet another important advantage of the process of this invention over the prior art is that it provides for the release of the hydrogen that is bound to the hydrogen acceptor when the saturated acceptor is subjected to a specific set of conditions in the regeneration vessel(s). Furthermore, the released hydrogen could be utilized to regenerate the catalyst or subjected to any other suitable chemical use or monetized to improve the overall aromatization process economics.

Another important advantage of the present invention is that it allows for different regeneration conditions to be used in the different regeneration vessel or vessels to optimize and minimize the regeneration time required for the catalyst and hydrogen acceptor and to improve performance in the aromatization reaction.

The aforementioned advantages of the process of the present invention provide for an efficient removal of hydrogen from the reaction zone of alkane-containing gas aromatization reactor operating in fluidized bed mode and for shifting the reaction equilibrium towards higher alkane-containing gas stream conversion and benzene yields per pass. Therefore, the present invention has the potential to allow for the commercialization of an economically attractive direct, non-oxidative C₂ to C₄ alkane aromatization process.

1. A process for the aromatization of alkanes in an alkane-containing gas stream, which alkane-containing gas stream contains at least one alkane selected from the group consisting of ethane, propane or butane and contains essentially no methane, comprising: contacting the alkane-containing gas stream in a reactor with a fluidized bed comprising an aromatization catalyst and a hydrogen acceptor under alkane aromatization conditions to produce a product stream comprising aromatics and hydrogen wherein the hydrogen is, at least in part, bound by the hydrogen acceptor in the reaction zone and removed from the product and reaction zone.

2. A process according to claim 1 wherein in the range of from 75% vol. to 100% vol. of the alkane in the alkane-containing gas is ethane.

3. A process according to claim 1, wherein the alkane conversion and corresponding benzene yield per pass are higher than the conversion and yield obtained with the same aromatization catalyst and under the same alkane aromatization conditions, but in the absence of a hydrogen acceptor in the reaction zone of the aromatization reactor.

4. A process according to claim 1, wherein the alkane-containing gas stream comprises carbon dioxide.

5. A process according to claim 1, wherein the aromatization catalyst comprises a zeolite selected from the group consisting of ZSM-5, ZSM-22, ZSM-8, ZSM-11, ZSM-12 or ZSM-35.

6. A process according to claim 1, wherein the aromatization catalyst comprises a metal selected from the group consisting of vanadium, chromium, manganese, zinc, iron, cobalt, nickel, copper, gallium, germanium, niobium, molybdenum, ruthenium, rhodium, silver, tantalum, tungsten, rhenium, platinum and lead and mixtures thereof.

7. A process according to claim 1, wherein the hydrogen acceptor comprises a metal or metals that are capable of selectively binding hydrogen under the alkane aromatization conditions in the reaction zone of a fluidized bed reactor.

8. A process according to claim 1, wherein the hydrogen acceptor comprises a metal selected from the group consisting of Ti, Zr, V, Nb, Hf, Co, Mg, La, Pd, Ni, Fe, Cu, Ag, Cr, Th and other transition metals and compounds or mixtures thereof.

9. A process according to claim 1, wherein the alkane aromatization conditions comprise a temperature in the range of from 480° C. to 700° C.

10. A process according to claim 1, further comprising continuously regenerating the catalyst to remove coke formed during the reaction and continuously regenerating the hydrogen acceptor by releasing the hydrogen under regeneration conditions.

11. A process according to claim 10 wherein the catalyst and hydrogen acceptor are regenerated in a single regeneration vessel.

12. A process according to claim 10, wherein the catalyst and hydrogen acceptor are regenerated in separate vessels

13. A process according to claim 10, wherein the catalyst and hydrogen acceptor are each regenerated under different regeneration conditions

14. A process according to claim 10, wherein the hydrogen released from the hydrogen acceptor is used for catalyst regeneration.

15. The process of claim 14 wherein supplemental hydrogen is supplied from an external source in order to properly complete the catalyst regeneration.

16. A process according to claim 10, wherein the hydrogen acceptor regeneration is accomplished under regeneration conditions including: feed rate, temperature and pressure that are substantially different from the alkane aromatization conditions

17. A process according to claim 10, wherein the hydrogen acceptor regeneration is accomplished with hydrogen containing off-gas from the aromatization reaction

18. A process according to claim 1, wherein the alkane-containing gas stream is derived from natural gas liquids
